(19) United States
(12) Patent Application Publication
(10) Pub. No.: US 2026/0305922 A1
(43) Pub. Date: Oct. 8, 2026

(54) ACTUATION MECHANISMS FOR MEDICAL DEVICES AND RELATED SYSTEMS

(71) Applicant: Boston Scientific Scimed, Inc., Maple Grove, MN (US)

(72) Inventors: Jonathan MELCHOR, Providence, RI (US); Cang Duy DAO, Attleboro, MA (US)

(73) Assignee: Boston Scientific Scimed, Inc., Maple Grove, MN (US)

(21) Appl. No.: 19/634,422

(22) Filed: Mar. 31, 2026

Related U.S. Application Data
(60) Provisional application No. 63/783,285, filed on Apr. 4, 2025.

(51) Int. Cl. A61B 1/005; A61B 1/00

(52) U.S. Cl. CPC A61B 1/0052; A61B 1/00042; A61B 1/0057

(57) ABSTRACT

Medical devices and actuation mechanisms thereof are described. The medical device may include a handle with an actuation mechanism comprising a cam, the cam including a plurality of posts on a surface of the cam. Each post of the plurality of posts may define a channel aligned with a channel of an adjacent post. The actuation mechanism may further include an actuator coupled to the cam and may be configured to receive and transmit user input to the actuation mechanism. The medical device may further include at least one control wire fixedly attached to the plurality of posts via the at least one channel of each post. The plurality of posts may be spaced apart from one another and may collectively provide a curved pathway for control wire(s), e.g., the curved pathway including a change in direction of less than 90 degrees.

### CROSS-REFERENCE TO RELATED APPLICATIONS

This application claims the benefit of priority to U.S. Provisional Application No. 63/783,285, filed on April 4, 2025, which is incorporated by reference herein in its entirety.

### TECHNICAL FIELD

Various aspects of the present disclosure relate generally to actuator mechanisms for medical devices. More specifically, this disclosure includes actuator mechanisms that include a plurality of fixing features disposed on a cam surface useful for enhanced control of control wires, among other aspects.

### BACKGROUND

Medical devices with deflectable distal tips have encountered challenges related to loss of deflection during medical procedures. This issue often stems from a phenomenon known as wire slip, in which the pull wire(s) controlling movement of the distal tip of the medical device becomes loose or shifts from its intended position within the device. A cause of wire slip can relate to the use of different materials. For example, securing metal pull wires to plastic components used in articulation control systems can present a notable challenge. Wire slip may compromise the performance and reliability of medical devices with deflectable tips. Wire slip may reduce the precision and range of tip deflection, potentially affecting a user's ability to navigate patient anatomy and to perform a medical procedure. Additionally, wire slip may lead to inconsistent deflection responses, which may impact the overall usability and effectiveness of the medical device.

### SUMMARY

This disclosure includes a medical device comprising a handle that includes an actuation mechanism, the actuation mechanism including a cam comprising a plurality of posts on a surface of the cam, wherein each post of the plurality of posts defines a channel aligned with a channel of an adjacent post. The actuation mechanism may further include an actuator coupled to the cam configured to receive and transmit user input to the actuation mechanism and/or at least one control wire fixedly attached to the plurality of posts via the at least one channel of each post. The posts may be spaced apart from one another and may collectively provide a curved pathway for the at least one control wire. The curved pathway may include a change in direction of less than 90 degrees.

Any of the devices disclosed herein may have any of the following features. The posts may include a first group of posts and a second group of posts, and the at least one control wire may include a first control wire and a second control wire. The first group of posts may include channels aligned with one another that may receive the first control wire extending along a first curved pathway, and the second group of posts may include channels aligned with one another that may receive the second control wire along a second curved pathway. The first curved pathway and the second curved pathway may intersect at a single position or point. The cam may comprise a polymer and the at least one control wire may comprise a metal or metal alloy. Optionally, the posts may be symmetric about a centerline of the cam. In some examples, the plurality of posts includes at least four posts. In some aspects, the plurality of posts may include at least one circular post and at least one elongated post spaced apart from the at least one circular post. The posts may include at least three circular posts spaced apart from one another. Further, for example, the at least one control wire may be welded to the plurality of posts. In some examples, the plurality of posts includes at least three circular posts and at least two elongated posts, each elongated post between two circular posts. In some aspects, the plurality of posts includes at least one post that defines two channels. In at least one example, the plurality of posts includes only one post that defines two channels. Optionally, the actuator may be or include a lever. In some aspects, the medical device further comprises a shaft extending from the handle, e.g., the at least one control wire extending distally through the shaft.

This disclosure also includes a medical device comprising a handle comprising an actuation mechanism that includes a cam, the cam including a plurality of posts on a surface of the cam, wherein each post of the plurality of posts defines a channel aligned with a channel of an adjacent post. The actuation mechanism may further include an actuator coupled to the cam and configured to receive and transmit user input to the actuation mechanism. Optionally, the medical device may further include a first control wire and a second control wire each fixedly attached to the plurality of posts. For example, the plurality of posts may include a first group of posts that includes channels that receive the first control wire extending along a first curved pathway, and a second group of posts that includes channels that receive the second control wire along a second curved pathway. The first curved pathway and the second curved pathway may intersect at a single post of the plurality of posts.

Any of the devices disclosed herein may have any of the following features. The cam may comprise a polymer and/or the at least one control wire may comprise a metal or metal alloy. The post at which the first curved pathway and the second curved pathway intersect may be a circular post, for example. In some aspects, the medical device further comprises a shaft extending from the handle, wherein the first control wire and the second control wire extend distally through the shaft.

This disclosure also includes a medical device comprising a handle comprising an actuation mechanism, the actuation mechanism including a cam comprising a plurality of posts on a surface of the cam, wherein each post of the plurality of posts defines a channel aligned with a channel of an adjacent post. The actuation mechanism may further include an actuator coupled to the cam and configured to receive and transmit user input to the actuation mechanism. The medical device may further include a first control wire and a second control wire each fixedly attached to the plurality of posts. For example, the plurality of posts may include a first group of posts that includes channels that receive the first control wire extending along a first curved pathway, and a second group of posts that includes channels that receive the second control wire along a second curved pathway. The first curved pathway and the second curved pathway may intersect at a single post of the plurality of posts. Each of the first curved pathway and the second curved pathway may include a change in direction of less than 90 degrees.

### BRIEF DESCRIPTION OF THE DRAWINGS

The accompanying drawings are incorporated in and constitute a part of this application. These drawings illustrate aspects of the disclosure that, together with the written description herein, serve to explain this disclosure. Each drawing depicts one or more exemplary aspects according to this disclosure, as follows:

FIG. 1 illustrates a side view of a medical device, according to aspects of this disclosure.

FIG. 2 illustrates a partial cutaway side view of a handle of the medical device of FIG. 1, showing internal articulation mechanism components, according to aspects of this disclosure.

FIG. 3 illustrates a side view of a cam of the articulation mechanism of the medical device of FIG. 1, according to aspects of this disclosure.

FIGS. 4A-4F and 5 illustrate additional exemplary articulation mechanism components useful for medical devices, according to aspects of this disclosure.

### DETAILED DESCRIPTION

Embodiments of the present disclosure may address one or more limitations in the art. The scope of the disclosure, however, is defined by the attached claims and not the ability to solve a specific problem.

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when placing the device into the subject. Proximal and distal directions are labeled with arrows marked “P” and “D,” respectively, throughout various figures.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

The medical devices may be used to perform a medical procedure at a desired treatment site. For example, as the treatment site may be any internal lumen, organ, cavity, or other tissue within the subject, including areas of the urinary system or the gastrointestinal system. Additionally, although ureteroscopes endoscopes are referenced herein as illustrative medical devices, it will be appreciated that the disclosure encompasses any medical device having an articulable distal end portion. In some examples, the medical device has a shaft that defines a lumen, e.g., a working channel, extending from a proximal end of the shaft to a distal end of the shaft. The medical device may include a ureteroscope, endoscope, duodenoscope, gastroscope, endoscopic ultrasonography (“EUS”) scope, colonoscope, bronchoscope, laparoscope, arthroscope, cystoscope, aspiration scope, sheath, or catheter. In some examples, the medical device may include a handle and a shaft extending distally from the handle. The shaft of the medical device may be useful in conjunction with another medical instrument, e.g., the shaft of the medical device being capable of being inserted into a working channel of the medical instrument (e.g., an endoscope or other type of scope). Alternatively, the shaft of the medical device may be used for a medical procedure following insertion directly into a subject.

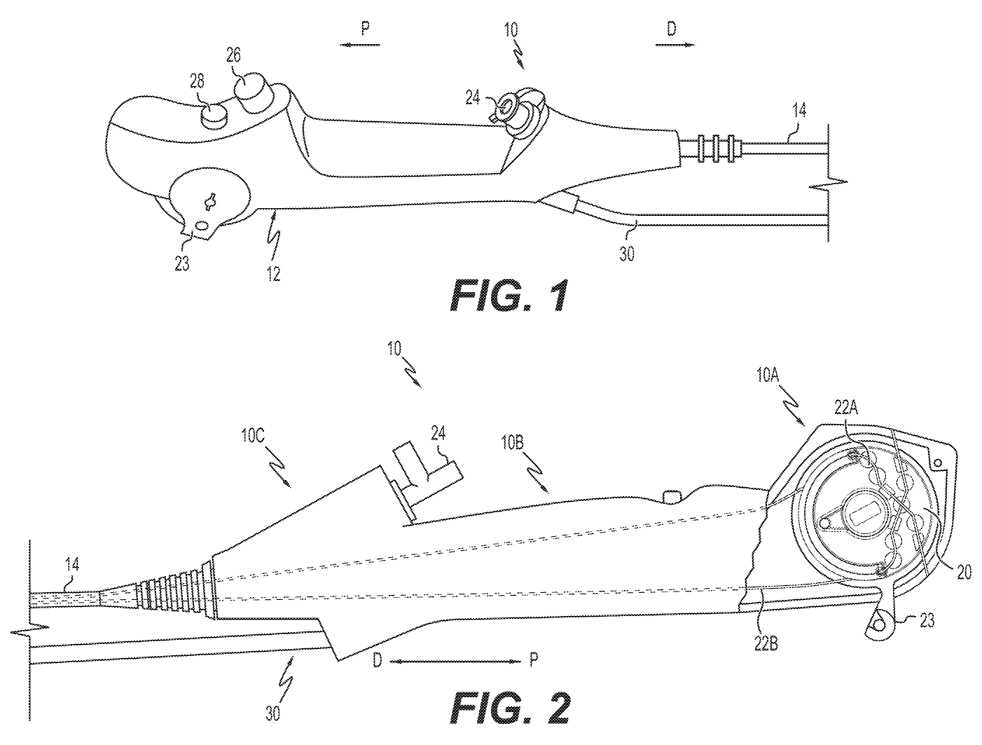

FIG. 1 depicts aspects of an exemplary medical device 10 according to the disclosure. FIG. 1 depicts a proximal portion of medical device 10, including a handle 12 and a shaft 14. Handle 12 may be configured for gripping and use by an operator. Shaft 14 may extend distally from handle 12. At least a portion of shaft 14 may be flexible, for example, to facilitate navigation through a subject’s anatomy, and shaft 14 may define one or more lumens, including a working channel and/or lumen(s) optionally containing wires, tubes, or other features passing therethrough. In some aspects, shaft 14 may include a single lumen. Shaft 14 may be configured for at least partial insertion into a body (e.g., a body lumen) of the subject.

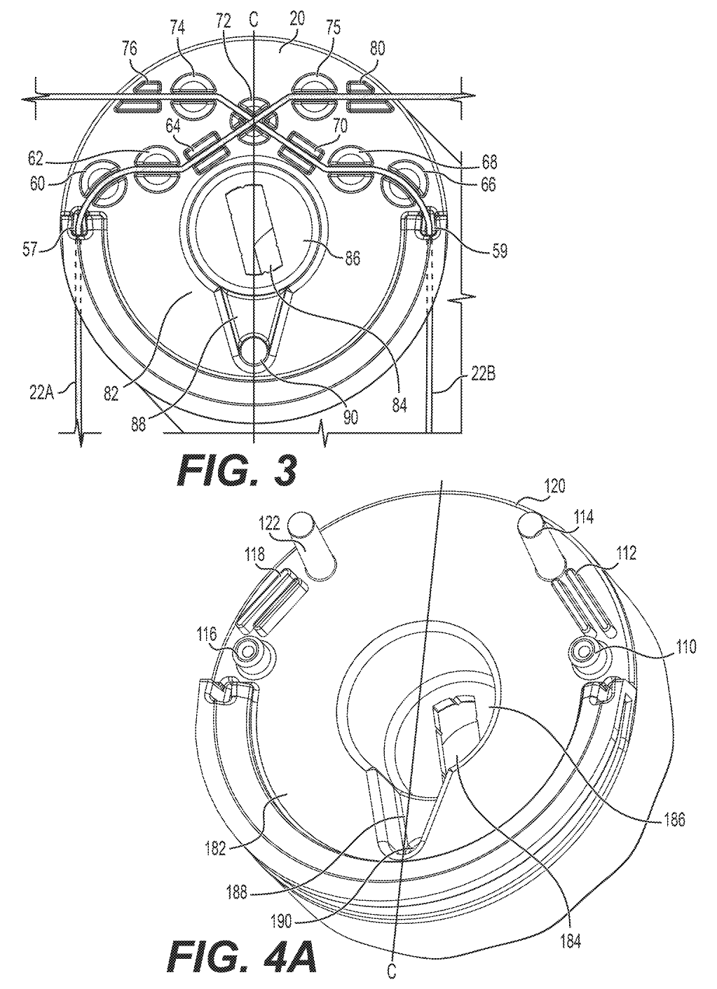

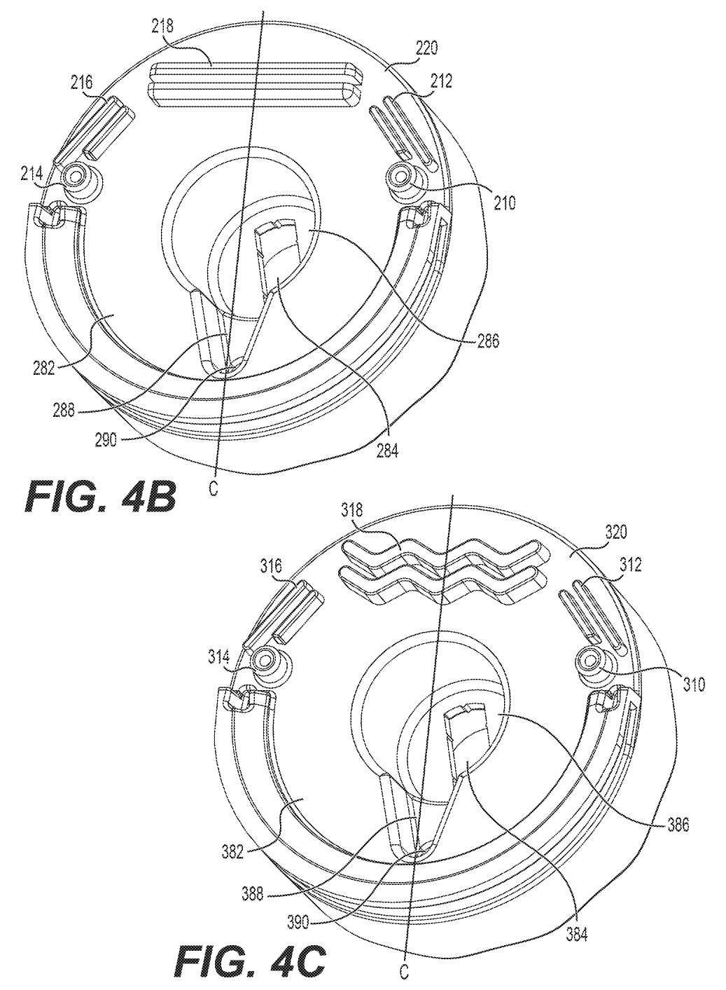

Medical device 10 may include one or more control wires (e.g., control wires 22A and 22B, shown in FIGS. 2 and 3) extending from handle 12 and through at least a portion of shaft 14. For example, the control wire(s) may extend longitudinally through shaft 14 (e.g., via one or more lumens of shaft 14). In some aspects, the control wire(s) may be coupled (e.g., directly or indirectly) to a distal portion of shaft 14. The control wire(s) may be configured to articulate the distal portion of shaft 14 in one or more directions. For example, the distal portion of shaft 14 may include an articulation portion, which may include one or more articulation joints within an outer sheath of shaft 14 coupled to the control wire(s).

Handle 12 may include a mechanism to control articulation of shaft 14. For example, the mechanism may include an actuator 23 such as a lever operably coupled to the control wire(s). For example, actuator 23 may be coupled to a proximal end and/or a proximal portion of the control wire(s) such that activation of actuator 23 controls articulation/steering of the distal end portion of shaft 14, including a distal tip of shaft 14. In some aspects, a proximal portion of handle 12 may include actuator 23 as shown in FIG. 1. It will be appreciated that any suitable actuator(s) may be used to transfer user input to actuation mechanism, such as one or more levers (as illustrated), knobs, buttons, sliders, switches, or joysticks. Handle 12 may include one or more locking mechanisms, for example, to lock a position of actuator 23 and, thus, lock an orientation of shaft 14 (e.g., a distal portion and/or distal tip).

A port 24 of handle 12 may provide access to a lumen (e.g., working channel) of shaft 14 of medical device 10. An operator may insert an instrument or other device into port 24 and may extend the instrument or other device distally through the working channel of 14. The working channel may extend longitudinally through a length of shaft 14, for example, and terminate at a distal opening of a distal tip of shaft 14. In some examples, handle 12 may include one or more valves. In the example illustrated, handle 12 includes a valve 26 on a proximal portion of handle 12, e.g., on an opposing side of handle 12 from actuator 23. An operator may connect handle 12 to a source of suction or fluid and may operate valve 26 to control suction or fluid through shaft 14 (e.g., through the working channel or other lumen, which may be the same or different from the lumen or working channel in communication with the port 24). Valve 26 may be, for example, a suction valve, fluid supply valve (e.g., air and/or water valve), and/or other type of valve that performs a combination of functions.

In some aspects, handle 12 may include an actuator operably coupled to an imaging device of shaft 14, e.g., to allow an operator to capture images. For example, as shown in FIG. 1, handle 12 includes a button 28 to allow the operator to capture still images and/or video images. While button 28 is shown on a proximal portion of handle 12, button 28 (or other actuator) may be on another part of handle 12 accessible to the operator’s hand during use. An umbilicus 30 may extend from handle 12 and may include or carry wires, cables, and/or conduits configured to provide, for example, power, signals, or fluids to and/or from handle 12. For example, umbilicus 30 may connect handle 12 to one or more user interfaces, monitors, control units, displays, etc.

FIG. 2 illustrates a partial cross-section of handle 12. Handle 12 comprises a proximal portion 10A, a distal portion 10C, and a central portion 10B. Proximal portion 10A may form a proximal-most end of handle 12, and distal portion 10C may form a distal portion of handle 12. Central portion 10B may be distal to proximal portion 10A and proximal to distal portion 10C. In some examples, proximal portion 10A and/or distal portion 10C may have a greater cross-sectional dimension (e.g., width) than that of central portion 10B. For example, proximal portion 10A and/or distal portion 10C may be wider than central portion 10B. In some examples, central portion 10B may be formed to facilitate the operator’s grip of handle 12. Proximal portion 10A, central portion 10B, and/or distal portion 10C may include one or more additional features to facilitate an operator’s grip on handle 12. For example, one or more additional features on proximal portion 10A, central portion 10B, and/or distal portion 10C may include surface features such as raised features and/or indented features, and/or may include a texture such as rough features and/or smooth features, or any combination thereof, to facilitate a user’s grip on handle 12. Shapes and relative sizes of handle 12 (e.g., the housing of handle 12) shown in the figures are merely exemplary, and other arrangements are contemplated within the scope of this disclosure. For example, in other examples, each of proximal portion 10A, central portion 10B, and distal portion 10C may have a same or similar cross-sectional dimension (e.g., width). In such a way, handle 12 may have the same cross-sectional width along substantially all of the longitudinal length of handle 12. For example, proximal portion 10A may have the same width as central portion 10B and distal portion 10C.

As mentioned above, handle 12 may include an actuation mechanism. For example, the actuation mechanism may include a cam coupled to one or more control wires. As shown in FIG. 2, a cam 20 may be disposed within the housing of handle 12, e.g., within proximal portion 10A of handle 12. Actuator 23 (e.g., a lever) may be coupled (e.g., directly or indirectly) to cam 20. For example, a suitable mechanical fastener such as, e.g., a screw, a press-fit, a bolt, a weld, a rivet, etc., may couple actuator 23 to cam 20. In some examples, actuator 23 may be integrally formed with cam 20. For example, cam 20 may be a single molded component including actuator 23. In such cases, at least a part of actuator 23 may be external to the housing of handle 12 and accessible to a user while cam 20 may be at least partially contained within the housing of handle 12. In other examples, handle 23 may be a separate component designed to mate with a corresponding portion of cam 20 (e.g., a first cavity 86, a second cavity 88, and/or a third cavity 90). In some aspects, cam 20 may have the shape of a disk.

A proximal end of each of one or more control wires, e.g., a control wire 22A and a control wire 22B, may be fixedly coupled to cam 20 via a plurality of posts. Optionally, control wires 22A, 22B may comprise a different material or combination of materials than cam 20 and/or the plurality of posts. For example, control wires 22A and 22B may comprise a metal or metal alloy, such as stainless steel or copper, while cam 20 and/or the plurality of posts thereof comprise a polymer. Exemplary polymers suitable for the cam 20 and/or other components of the actuation mechanism (e.g., actuator 23, etc.) include, but are not limited to, thermoplastics such as acrylonitrile butadiene styrene (ABS). As described in further detail below with respect to FIG. 3, each of the one or more control wires may be coupled to a proximal portion of cam 20. The proximal portions of each of the one or more control wire(s) may be coupled to cam 20 via the plurality of posts. For example, control wires 22A and 22B may be affixed to cam 20 via welding, e.g., heat stake welding, laser welding, and/or ultrasonic welding, or by other suitable mechanisms such as a screw and nut.

A distal end each control wire may be coupled (e.g., directly or indirectly) to a distal portion of shaft 14. For example, each control wire may extend distally through handle 12 and shaft 14 to a distal portion of shaft 14. Accordingly, as actuator 23 is moved in different directions (e.g., rotated clockwise or counterclockwise), each of control wires 22A and 22B may be translated proximally or distally. For example, as actuator 23 rotates clockwise such that cam 20 rotates clockwise, control wire 22A is translated distally and control wire 22B is translated proximally. The distal translation of control wire 22A and proximal translation of control wire 22B may in turn move a distal portion of shaft 14, as previously described with respect to FIG. 1. For example, the distal translation of control wire 22A and the proximal translation of control wire 22B may bend an articulation section of shaft 14 in a first direction (e.g., upward).

Furthermore, as actuator 23 is rotated counterclockwise, cam 20 rotates such that control wire 22A is translated proximally and control wire 22B is translated distally. The proximal translation of control wire 22A and the distal translation of control wire 22B similarly may move shaft 14. For example, the proximal translation of control wire 22A and the distal translation of control wire 22B may bend an articulation section of shaft 14 in a second direction opposite the first direction (e.g., downward).

FIG. 3 illustrates a side view of a cam 20 of the actuation mechanism of the medical device shown in FIGS. 1 and 2. As mentioned above, cam 20 optionally may have the shape of a disk, e.g., having a circular body with a plurality of posts arranged in a pattern on its surface 82. All or some of the posts may be spaced apart from one another. In this example, the plurality of posts includes circular posts 60, 62, 72, 75, 66, 68, and 74 (e.g., having a generally circular cross-section); as well as elongated posts 64, 80, 70, 76. The elongated posts may have a generally square, rectangular, trapezoidal, or other non-circular cross-sectional shape. It should be understood that the shapes of posts shown are illustrative; other examples encompassed by the present disclosure include posts that may have other shapes. At least some and optionally all of the posts may define at least one channel. When a post defines a channel therein, the channel may extend from one side of the post to another side of the post.

For the example illustrated in FIG. 3, each post defines at least one channel therein. In some examples, the post defines a single channel (e.g., posts 60, 62, 75, 66, 68, 74, 64, 80, 70, 76). Optionally, one or more posts of the plurality of posts may define more than one channel, e.g., two channels (e.g., post 72). The plurality of posts is arranged to provide one or more pathways to receive and secure at least one control wire, e.g., via channels of posts that are aligned with each other. The plurality of posts may be arranged symmetrically on surface 82, e.g., with respect to a centerline C.

Cam 20 may include an opening to house a corresponding control wire extending proximally through handle 12 for coupling to cam 20. For example, cam 20 may include openings 57, 59 that receive corresponding control wires 22A, 22B. The example illustrated in FIG. 3 is designed to accommodate two control wires 22A, 22B. In other examples, the actuation mechanism may be designed to accommodate a single control wire (e.g., only control wire 22A or only control wire 22B). In such cases, the single control wire may be useful to deflect the shaft of a medical device (e.g., shaft 14 of medical device 10) along a single plane. For actuation mechanisms that include two control wires (e.g., control wires 22A and 22B), the two control wires may be useful to deflect the shaft of a medical device (e.g., shaft 14 of medical device 10) along two different planes.

Still referring to FIG. 3, a first pathway may be curved, the first pathway receiving control wire 22A. The first curved pathway may be defined by aligned channels of posts 60, 62, 64, 72, 75, and 80. A second pathway may be curved, the second pathway receiving control wire 22B. The second curved pathway may be defined by aligned channels of posts 66, 68, 70, 72, 74, and 76. The two control wires 22A, 22B intersect at a single position, of post 72. As mentioned above, post 72 is an example that defines two channels capable of receiving control wire 22A extending in a first direction, and control wire 22B extending in a second direction different from the first direction.

The arrangement of the plurality of posts may provide pathways, e.g., curved pathways (including tortuous pathways), for control wires that avoid sharp turns that could contribute to wire slip over time. For example, the pathways may be curved and include a change in direction of 90 degrees or less. This gentle curvature may allow for a smooth cycling of wire tension before the control wires are fixed to the cam (e.g., via welding) and may provide for consistent tension while being secured to the cam (e.g., via welding).

In FIG. 3, control wire 22A follows gentle changes in directions from the channel of post 60 to the channel of post 62, from the channel of post 62 to the channel of post 64, from the channel of post 64 to the channel of post 72, from the channel of post 72 to the channel of post 75, and from the channel of post 75 to the channel of post 80. As control wire 22A extends from the channel of one post to the next aligned channel, the change in direction of the control wire 22A is less than 90 degrees. For example, for the portion of pathway from post 72 to 75, the angle of control wire 22A forms an obtuse angle because the change in direction is less than 90 degrees. Control wires 22A and 22B may be fixed to each post that defines the pathway along which each extends, e.g., via welding. As each control wire 22A, 22B is welded to cam 20, material flow in the channels may create interference between each control wire 22A, 22B and cam 20. In some aspects, the attachment between cam 20 and each control wire 22A, 22B may be stronger than the tensile strength of the respective control wire 22A, 22B (e.g., an average tensile strength of 16 lbs). Optionally, control wires 22A and 22B may have a low friction coating along at least a portion of their surfaces to reduce friction, e.g., at the intersection position or point (e.g., post 72 in FIG. 3). One control wire 22A or 22B may pass over or under the other control wire 22A or 22B at the intersection position or point. In this example, control wires 22A, 22B only share the weld at one location, and the rest of the welded locations of each control wire 22A, 22B are independent of the welded locations of the other control wire 22A, 22B.

In some aspects, cam 20 may include engagement features configured to facilitate the attachment and operation of actuator 23. For example, cam 20 may include a first cavity 86, a second cavity 88, and a third cavity 90. The cavities may be configured to receive correspondingly shaped portions of actuator 23, facilitating a secure connection between cam 20 and actuator 23. This arrangement translates movement from actuator 23 (following input from a user) to cam 20, which in turn may control the articulation of the distal portion of shaft 14 via control wires 22A, 22B.

In some cases, the engagement features of cam 20 may be centrally located. For example, first cavity 86, second cavity 88, and third cavity 90 may be bisected by centerline C of cam 20. First cavity 86 may be approximately circular and be positioned at the center of surface 82. First cavity 86 may include an opening 84. Opening 84 may be approximately rectangular and may be configured to accept a correspondingly shaped portion of actuator 23. Adjacent to first cavity 86 is second cavity 88, which has an approximately trapezoidal shape. Second cavity 88 may be configured have a lesser depth than first cavity 86. In other words, second cavity 88 may be closer to viewing plane of FIG. 3 than first cavity 86. Second cavity 88 may be adjacent to third cavity 90 at a distal end of second cavity 88. Third cavity 90 may be approximately circular and may have a greater depth than second cavity 88. In other words, third cavity 90 may be positioned further from the viewing plane of FIG. 3 than second cavity 88. In some aspects, second cavity 88 may be of intermediate depth as compared to first cavity 86 and third cavity 90.

The arrangement of first cavity 86, second cavity 88, and third cavity 90 forms a composite shape on cam 20 as viewed from the side of cam 20. This configuration may include a circular portion of first cavity 86 distally transitioning into the tapered trapezoidal section of second cavity 88, which itself terminates in the circular region of third cavity 90. The arrangement of these cavities may allow cam 20 to interface with correspondingly shaped features on actuator 23.

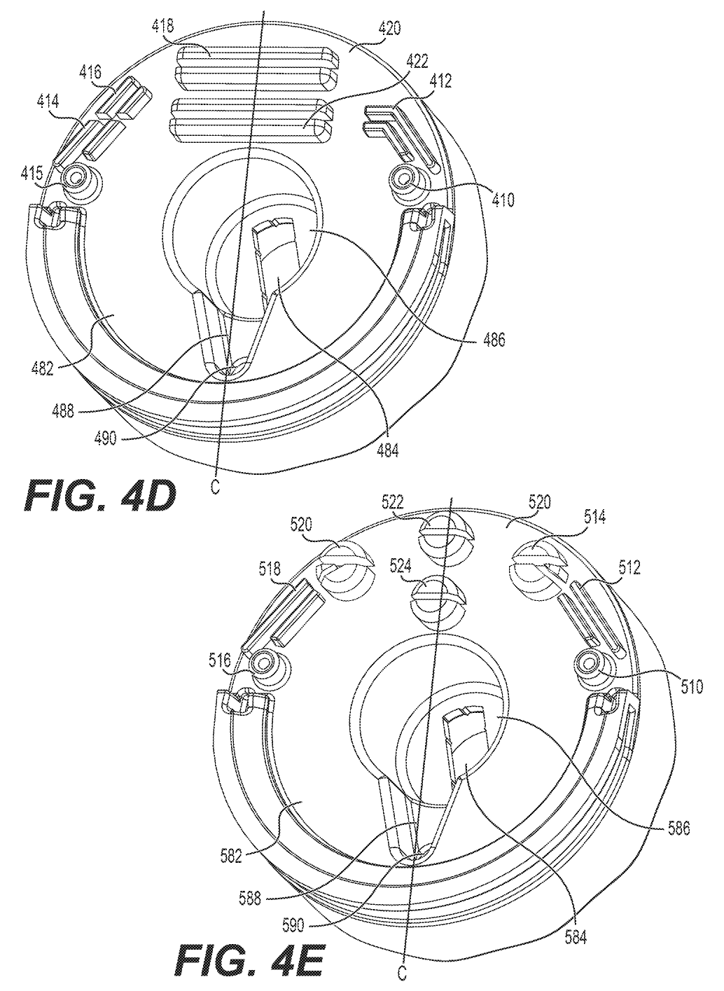

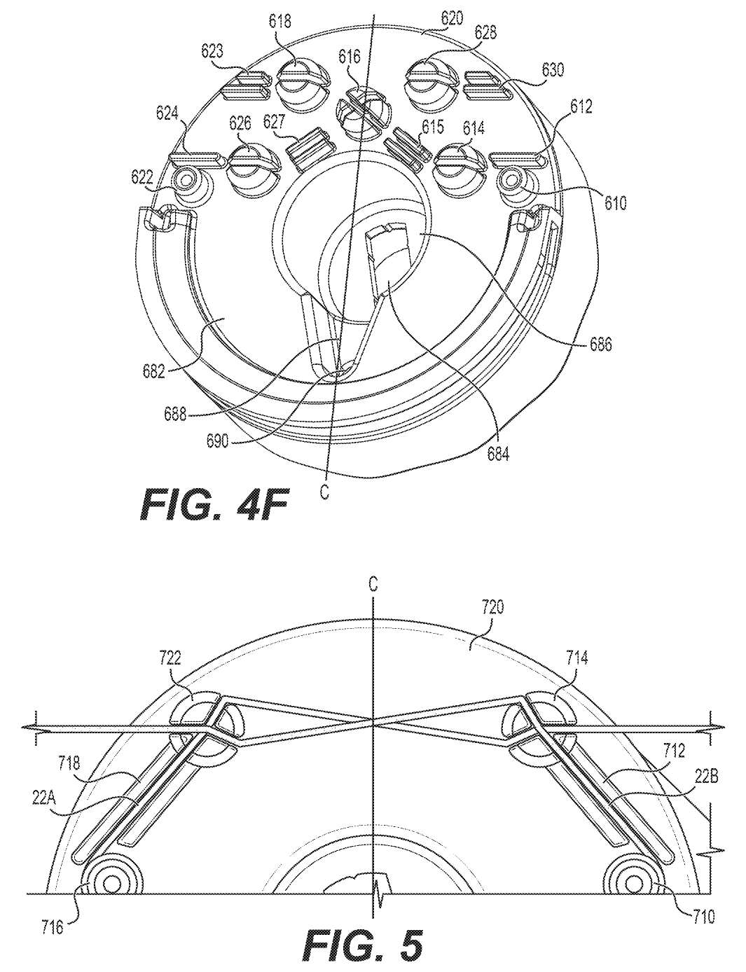

Additional examples of actuation mechanisms are illustrated in FIGS. 4A-4F and 5. Similar to the example of FIG. 3, FIGS. 4A-4F and 5 show examples of cams (cam 120, 220, 320, 420, 520, 620, and 720, respectively) that include a plurality of posts arranged to provide a more robust attachment of one or more control wires to the cam, e.g., to inhibit or avoid wire slip over time. Unless otherwise specified, the actuation mechanisms illustrated in FIGS. 4A-4F and 5 may include any of the features of the actuation mechanism illustrated in FIG. 3, and may be used in conjunction with any of the components and/or features of medical device 10 shown in FIGS. 1 and 2. While the additional examples shown in FIGS. 4A-4F and 5 are designed to accommodate two control wires (e.g., control wires 22A and 22B similar to FIG. 3), it is understood that any of these examples may instead accommodate a single control wire, e.g., by using only one of the two pathways described and optionally omitting posts that do not define the one pathway. It is understood that in some aspects, multiple control wires may share at least a portion of one or more posts. Further, in these examples, the material of the control wire(s) (e.g., metal or metal alloy) may be different from the material of the cam and posts thereof (e.g., comprising a polymer such as ABS). The control wire(s) may be fixedly attached to the post of the cam surface, e.g., via welding.

In FIG. 4A, cam 120 has a surface 182 with a plurality of posts, two of which define channels therein (posts 118 and 112) and four of which do not define channels (posts 116, 122, 110, and 114). As in the example of FIG. 3, the posts are spaced apart from one another and provide curved pathways for control wires having a change in direction of less than 90 degrees. For example, a first control wire (e.g., control wire 22A of FIG. 3) may wrap around at least part of the circumference of post 116, extend through the channel of post 118, wrap around at least part of the circumference of post 112, and extend across surface 182 to wrap around at least part of the circumference of post 114. Similarly, a second control wire (e.g., control wire 22B of FIG. 3) may wrap around at least part of the circumference of post 110, extend through the channel of post 112, wrap around at least part of the circumference of post 114, and extend across surface 182 to wrap around at least part of the circumference of post 122. The control wires may be fixed to each respective post, e.g., via welding. Cam 120 may include a first cavity 186, a second cavity 188, and a third cavity 190 as engagement features configured to receive corresponding engagement features of an actuator as discussed above in connection to FIG. 3.

FIG. 4B illustrates another exemplary cam 220 of an actuation mechanism according to the present disclosure. A surface 282 of cam 220 includes a plurality of posts, three of which define channels therein (posts 216, 212, and 218), and two of which do not define channels (posts 214 and 212). The posts are spaced apart from one another and provide curved pathways for control wires, providing each control wire with a change in direction of less than 90 degrees. For example, a first control wire (e.g., control wire 22A of FIG. 3) may wrap around at least part of the circumference of post 214, extend through the channel of post 216, and extend through the channel of post 218. Similarly, a second control wire (e.g., control wire 22B of FIG. 3) may wrap around at least part of the circumference of post 210, extend through the channel of post 212, and extend through the channel of post 218, e.g., adjacent to the first control wire within the channel of post 218. The channel of post 216 may be aligned with one side of the channel of post 218 so as to provide a gentle curving path for the first control wire, and the channel of post 212 similarly may be aligned with the opposite side of the channel of post 218 to provide a gentle curving path for the second control wire. The control wires may be fixed to each respective post, e.g., via welding. Cam 220 may include a first cavity 286, a second cavity 288, and a third cavity 290 as engagement features configured to receive corresponding engagement features of an actuator as discussed above in connection to FIG. 3.

FIG. 4C illustrates another example similar to that of FIG. 4B but providing a someone more tortuous pathway via a post 318 serving a function similar to post 218. Cam 320 includes a surface 382 with a plurality of posts three of which define channels therein (posts 316, 312, and 318), and two of which do not define channels (posts 314 and 312). The plurality of posts is arranged similarly to that of cam 220 discussed above, except the channel of post 318 is curved in a zigzag direction, whereas the channel of post 218 above is straight. The curved shape of the channel may provide for a more secure attachment (e.g., via welding) of the two control wires adjacent to each other within the channel of post 318. Cam 320 may include a first cavity 386, a second cavity 388, and a third cavity 390 as engagement features configured to receive corresponding engagement features of an actuator as discussed above in connection to FIG. 3.

While each of the examples shown in FIGS. 3 and 4A-4C include a plurality of posts in a symmetric arrangement (e.g., about a centerline C of the respective cams), FIG. 4D illustrates an example without symmetry about a centerline C. This arrangement provides the option of securing control wires to separate posts without an intersection position or point. The cam 420 includes a surface 482 with a plurality of posts, five of which define channels therein (posts 414, 416, 418, 412, and 422), and two of which do not define channels (posts 415 and 410). The posts are spaced apart from one another and provide curved pathways for control wires, providing each control wire with a change in direction of less than 90 degrees. For example, a first control wire (e.g., control wire 22A of FIG. 3) may wrap around at least part of the circumference of post 415, extend through the channel of post 414, extend through the channel of post 416, and then extend through the channel of post 418. That is, the channels of posts 414, 416, and 418 may be aligned and collectively form a curved pathway for the first control wire. Similarly, a second control wire (e.g., control wire 22B of FIG. 3) may wrap around at least part of the circumference of post 410, extend through the channel of post 412, and extend through the channel of post 422. That is, the channel of post 412 may be aligned with the channel of post 422 to provide a gentle curvature between the two. While the channels of posts 414 and 416 are straight, the channel of post 412 has an obtuse angle that provides a change in direction for the second control wire of less than 90 degrees. The angled shape of post 412 provides for alignment with the channel of post 422 rather than that of post 418. In such cases, the control wires may be fixedly attached to separate posts, e.g., via welding. Cam 420 may include a first cavity 486, a second cavity 488, and a third cavity 490 as engagement features configured to receive corresponding engagement features of an actuator as discussed above in connection to FIG. 3.

FIG. 4E shows yet another exemplary cam 520 having a surface 582 with a plurality of posts. In this example, the plurality of posts includes six posts that define channels therein (posts 518, 519, 522, 512, 514, and 524) and two posts that do not define channels (posts 516 and 510). Like the examples of FIGS. 3 and 4A-4C, the arrangement of posts is symmetric about a centerline C of the surface 582. The posts are spaced apart from one another and provide curved pathways for control wires, providing each control wire with a change in direction of less than 90 degrees. For example, a first control wire (e.g., control wire 22A of FIG. 3) may wrap around at least part of the circumference of post 516, extend through the channel of post 518, extend through the channel of post 519, and then through the channel of post 522 or 524. Similarly, a second control wire (e.g., control wire 22B of FIG. 3) may wrap around at least part of the circumference of post 510, extend through the channel of post 512, extend through the channel of post 514, and then through the channel of post 522 or 524. For example, if the first control wire extends through post 522, the second wire may extend through post 524, such that the two control wires are not attached to the same post. That is, the pathway of the first control wire may be defined by post 516, the channel of post 518, the channel of post 519 and the channel of post 522 and/or post 524. Similarly, the pathway of the second control wire may be defined by post 510, the channel of post 512, the channel of post 514, and the channel of post 522 and/or post 524. Cam 520 may include a first cavity 586, a second cavity 588, and a third cavity 590 as engagement features configured to receive corresponding engagement features of an actuator as discussed above in connection to FIG. 3.

FIG. 4F illustrates a cam 620 with a plurality of posts on its surface 682, in an arrangement similar to that of cam 20 of FIG. 3. In this example, the plurality of posts includes nine posts that define channels therein (posts 626, 627, 616, 618, 623, 614, 615, 628, and 630) and four posts that do not define channels therein (posts 622, 624, 610, and 612). The main difference compared to the example of FIG. 3 is replacing post 60 of FIG. 3 (defining a channel therein from one side of post 60 to the opposite side of post 60) with post 622 (without a channel); and replacing post 66 of FIG. 3 with post 610. That is, a control wire may wrap around at least part of the circumference of post 622 before extending through the channel of post 626 and continuing along the pathway, similar to the example of FIG. 3. Another post 624 is adjacent to post 622, e.g., providing a guiding surface opposite post 622. Post 624 may help to follow the intended pathway, e.g., to avoid inadvertently routing a control wire through a post other than post 626 (e.g., post 618) after the control wire wraps around post 622. Similarly, post 612 may inhibit inadvertently routing a control wire through a post other than post 614 after the wire wraps around post 610. Cam 620 may include a first cavity 686, a second cavity 688, and a third cavity 690 as engagement features configured to receive corresponding engagement features of an actuator as discussed above in connection to FIG. 3.

FIG. 5 illustrates another exemplary cam 720 useful in actuation mechanisms of medical devices according to the present disclosure. In this example, the surface 782 of cam 720 includes a plurality of six posts, including four posts that define channels therein (posts 718, 722, 712, and 714) and two posts that do not define channels (posts 716 and 710). In contrast to previous examples, the posts are not spaced apart from one another. Also, this arrangement of posts provides for multiple intersections of two control wires. A first control wire 22A extends along a curved pathway defined by wrapping around at least part of the circumference of post 716, extending through the channel of post 718, extending through one of two channels defined by post 722, and extending through one of two channels defined by post 714. Similarly, a second control wire 22B extends along a curved pathway defined by wrapping around at least part of the circumference of post 710, extending through the channel of post 712, extending through the other of two channels defined by post 714, and extending through the other of two channels defined by post 722. In addition to intersecting in posts 722 and 714, the two control wires 22A, 22B intersect at a midpoint between posts 722 and 714 (e.g., along centerline C of cam 720). As with the other examples, the pathways for control wires 22A, 22B provide gentle curves that avoid sharp changes of direction, e.g., to promote secure attachment and avoid wire slip over time.

The actuation mechanisms herein may facilitate assembly and/or dependability over time. For example, the pathways provided by the plurality of posts that avoid sharp changes in direction may provide the ability to provide consistent tension to control wires as the control wires are fixed to the cam surface, e.g., via welding.

While principles of the disclosure are described herein with reference to illustrative aspects for particular medical procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

1. A medical device comprising:
a handle comprising an actuation mechanism that includes:
a cam comprising a plurality of posts on a surface of the cam, wherein each post of the plurality of posts defines a channel aligned with a channel of an adjacent post;
an actuator coupled to the cam and configured to receive and transmit user input to the actuation mechanism; and
at least one control wire fixedly attached to the plurality of posts via the at least one channel of each post,
wherein the plurality of posts are spaced apart from one another and collectively provide a curved pathway for the at least one control wire, the curved pathway including a change in direction of less than 90 degrees.

2. The medical device of claim 1, wherein the plurality of posts includes a first group of posts and a second group of posts, and wherein the at least one control wire includes a first control wire and a second control wire, the first group of posts including channels aligned with one another that receive the first control wire extending along a first curved pathway, and the second group of posts including channels aligned with one another that receive the second control wire along a second curved pathway.

3. The medical device of claim 2, wherein the first curved pathway and the second curved pathway intersect at a single position.

4. The medical device of claim 1, wherein the cam comprises a polymer and the at least one control wire comprises a metal or metal alloy.

5. The medical device of claim 1, wherein the plurality of posts is symmetric about a centerline of the cam.

6. The medical device of claim 1, wherein the plurality of posts includes at least four posts.

7. The medical device of claim 1, wherein the plurality of posts includes at least one circular post and at least one elongated post spaced apart from the at least one circular post.

8. The medical device of claim 1, wherein the plurality of posts includes at least three circular posts spaced apart from one another.

9. The medical device of claim 1, wherein the at least one control wire is welded to the plurality of posts.

10. The medical device of claim 2, wherein the plurality of posts includes at least three circular posts and at least two elongated posts, each elongated post being between two circular posts.

11. The medical device of claim 10, wherein the first curved pathway and the second curved pathway intersect at one point.

12. The medical device of claim 1, wherein the plurality of posts includes at least one post that defines two channels.

13. The medical device of claim 12, wherein the plurality of posts includes only one post that defines two channels.

14. The medical device of claim 1, wherein the actuator is a lever.

15. The medical device of claim 1, further comprising a shaft extending from the handle, wherein the at least one control wire extends distally through the shaft.

16. A medical device comprising:
a handle comprising an actuation mechanism that includes:
a cam comprising a plurality of posts on a surface of the cam, wherein each post of the plurality of posts defines a channel aligned with a channel of an adjacent post;
an actuator coupled to the cam and configured to receive and transmit user input to the actuation mechanism; and
a first control wire and a second control wire each fixedly attached to the plurality of posts,
wherein the plurality of posts includes a first group of posts that includes channels that receive the first control wire extending along a first curved pathway, and a second group of posts that includes channels that receive the second control wire along a second curved pathway; and
wherein the first curved pathway and the second curved pathway insect at a single post of the plurality of posts.

17. The medical device of claim 16, wherein the cam comprises a polymer and the at least one control wire comprises a metal or metal alloy.

18. The medical device of claim 16, wherein the post at which the first curved pathway and the second curved pathway intersect is a circular post.

19. The medical device of claim 16, further comprising a shaft extending from the handle, wherein the first control wire and the second control wire extend distally through the shaft.

20. A medical device comprising:
a handle comprising an actuation mechanism that includes:
a cam comprising a plurality of posts on a surface of the cam, wherein each post of the plurality of posts defines a channel aligned with a channel of an adjacent post;
an actuator coupled to the cam and configured to receive and transmit user input to the actuation mechanism; and
a first control wire and a second control wire each fixedly attached to the plurality of posts,
wherein the plurality of posts includes a first group of posts that includes channels that receive the first control wire extending along a first curved pathway, and a second group of posts that includes channels that receive the second control wire along a second curved pathway;
wherein the first curved pathway and the second curved pathway insect at a single post of the plurality of posts; and
wherein each of the first curved pathway and the second curved pathway includes a change in direction of less than 90 degrees.
